(19) United States
(12) Patent Application Publication
(10) Pub. No.: US 2010/0152146 A1
(43) Pub. Date: Jun. 17, 2010

(54) Topical Administration of Danazol

(75) Inventors: Paul Dmowski, Oak Park, IL (US); Gerianne T. DiPiano, Malvern, PA (US)

Correspondence Address: Pabst Patent Group LLP, 1545 PEACHTREE STREET NE, SUITE 320, ATLANTA, GA, 30309, US

(73) Assignee: FemmePharma Holding Company, Inc.

(21) Appl. No.: 12/712,497

(22) Filed: Feb. 25, 2010

Related U.S. Application Data
(62) Division of application No. 11/964,357, filed on Dec. 26, 2007.
(60) Provisional application No. 60/871,889, filed on Dec. 26, 2006.

(51) Int. Cl. A61K 31/56; A61P 3/00; A61P 17/00

(52) U.S. Cl. 514/178

(57) ABSTRACT

Pharmaceutical preparations for topical or local administration of drugs directly to the skin for treatment of disorders of the subcutaneous fatty tissue, in particular in cases of cellulite, are disclosed herein. In a preferred embodiment, the drug is danazol or gastrinone. In another embodiment, the drug is danazol in combination with an aromatase inhibitor or an estrogen compound. The preferred formulations contain drugs in the form of micro or nanoparticles, which may be formed of drug alone or in combination with an excipient or carrier. The excipient or carrier may modify the release rates or enhance absorption into the affected area. The drug formulation may be in the form of a cream, lotion, ointment, gel or emulsion, solution or foam.

### CROSS-REFERENCE TO RELATED APPLICATIONS

This application claims priority to U.S. Ser. No. 60/871,889, filed Dec. 26, 2006.

### FIELD OF THE INVENTION

The present invention relates to pharmaceutical preparations containing danazol or other testosterone analogs, which can be administered topically to treat disorders of the subcutaneous tissue, particularly cellulite.

### BACKGROUND OF THE INVENTION

Compositions and methods of tinning and smoothing the skin are an important cosmetic challenge. An undesirable consequence of the formation of fatty tissue in the skin is, in particular, cellulite.

Cellulite is a term for non-inflammatory constitutional (gender-typical) adiposis with mild lymphatic blockade and mild (mucoid) formation of edema in the connective tissue zone (so-called Adipositas circumscripta oedematosa). Cellulite is found in particular in women in the hip, thigh and gluteal region. In most cases, a so-called “quilt syndrome” (connective tissue septation resulting in reticulate dimpling of the surface) and the so-called “orange-peel skin syndrome” (infundibuliform follicular retractions after squeezing) results. This results in connective tissue disorder of the subcutis and an increase in the bulk of lipids in the fat cavities. However, cellulite symptoms are not pathological.

Some cellulite treatments have focused on reconstituting sufficient vascularization of, and supply to, the dermis to target the reduced function of the vascular system which represents the major damage held responsible for the formation of cellulite. To this end, massage systems have been developed which include deep heating by applying electromagnetic waves (U.S. Pat. No. 5,778,894 to Dorogi, et al.). Besides massage systems, numerous treatments have targeted lypolysis to reduce the bulk of lipids in fat cavities. Lipolysis or fat breakdown occurs when hormone sensitive lipase (HSL) is activated. HSL activation requires phosphorylation via a cAMP (cyclic adenosine monophosphate) dependent protein Kinase. cAMP is therefore rate limiting in lipolysis. Net cAMP level depends on a balance between its enzymatic synthesis via adenylate cyclase, and its breakdown via phosphodiesterase. Adipocytes express both beta receptors which activate, and alpha-2 receptors which inactivate adenylate cytase. Creams targeting this pathway have recently been marketed that include xanthine based adenosine (a potent endogenous inhibitor of lipolysis) antagonists and phosphodiesterase inhibitors such as caffeine or theophylline, beta adrenergic agonists-isopreterenol (U.S. Pat. No. 4,588,724 to Greenway, III, et at) which stimulates adenylate cyclase to increase cAMP levels, and alpha-2-adrenergic antagonists-yohimbine (U.S. Pat. No. 4,588,724 to Greenway, III, et at; U.S. Pat. No. 4,524,359 to Champagne), Ginko bibola (U.S. Pat. No. 5,194,259 to Soudant, et al.), Chinese herbs (U.S. Pat. No. 5,77,894 to Dorogi, et al.) and extracts of a Malvaceae plant (U.S. Pat. No. 5,705,170 to Kong, et al.), to block antilipolytic inactivation of adenylate cyclase. Other compositions targeting cellulite contain inositol phosphate, to improve collagen synthesis (U.S. Pat. No. 5,536,499 to Znaiden, et al.). Another cream for treatment of cellulite contains, as active ingredients, extracts of Elizabethae, a coral species, and of heather, to combat inflammation in the tissue and thus the formation of tissue-weakening enzymes, in addition to an algal constituent intended to inactivate lipid oxidation. Centella asiatica, milk proteins and vitamin A are also intended to promote the weakened collagen and elastin production, and fruit acids are intended to smooth the skin. A topical composition containing a sugar compound that is converted into glycosoaminoglycan to thicken the skin; a primary antioxidant to inhibit formation of collagenase or elastase; an amino acid to thicken the skin and at least one transition metal to bind collagen and elastic fibers and thicken the skin is described in U.S. Pat. No. 6,358,539 to Murad. Others have attempted to use certain actives to reduce cellulite. U.S. Pat. No. 5,945,109 to Schmidt, et al., and U.S. Pat. No. 6,071,526 to Schmidt, et al. describe compositions and methods which include aromatase inhibitors and/or anti-estrogens for treatment of cellulite. None of these formulations have been established as a satisfactory treatment for cellulite.

The mechanical methods used to treat cellulite such as massage irritates cells, which as a result produce more elastase and collagenase, which in turn degrade connective tissue, allegedly tending to make it go limp rather than firm.

It is therefore an object of the present invention to provide a topical formulation to treat disorders of the subcutaneous connective fatty tissue, in particular, cellulite, so as to reduce the abundance and/or appearance of cellulite.

It is still another object of the present invention to provide a topical composition and a method of administration of the composition with diminished side effects as compared to formulations administered systemically.

BRIEF SUMMARY OF THE INVENTION

Topical formulations of danazol or other testosterone analogs are used in the treatment of disorders of the subcutaneous connective fatty tissue, in particular, treatment of cellulite. In a preferred embodiment, a locally or regionally effective amount of danazol is formulated as a crème, lotion, ointment, emulsion, shea butter, gel, suspension, solution or transdermal patch, and is applied in or adjacent to the area to be treated. The presence and appearance of cellulite is reduced after administration of the composition containing danazol. These compositions and methods for administration thereof provide for significantly diminished side effects as compared to systemic administration of the active ingredients while still reducing the abundance and/or appearance of cellulite.

### DETAILED DESCRIPTION OF THE INVENTION

I. Formulations

The formulation is designed to provide dispersal in the affected tissues with dissemination throughout the affected area to be treated, with little to no increase in systemic blood levels of the drug. The formulations can consist solely of drug, or drug combined with one or more excipients, preferably for topical transdermal administration. The formulation can also further include other constituents such as penetration enhancers or other active ingredients. The preferred formulations contain drugs in the form of micro or nanoparticles, which may be formed of drug alone or in combination with an excipient or carrier. The drug formulation may be in the administered as a crème, lotion, ointment, emulsion, shea butter, gel, suspension, solution or transdermal patch.

A. Testosterone and GnRH Analogs

Danazol and other Testosterone Analogs

In a preferred embodiment, the drug is danazol or gestrinone or another testosterone analog. Danazol is an isoxazolo derivative of 17ethenyltestosterone (an androgen hormone), a synthetic steroid analog which inhibits midcycle LH and FSH surge from the pituitary gland, thereby suppressing ovarian hormone production when administered systemically. In another embodiment, the drug includes danazol in combination with an aromatase inhibitor or anti-estrogen compound, such as those described in U.S. Pat. No. 6,071,526 to Schmidt, or progesterone-receptor antagonists. Progesterone Receptor Antagonists RTI 3021-012 and RTI 3021-022 are described by Wagner, et al. Endocrinology 140(3):1449-1458 (1999) and 6-aryl benzimidazolones and benzothiazolones by Zhang, et al., Bioorganic and Medicinal Chemistry Letters 11(2), 2747-2750 (2001).

Other GnRH-analogs that may be useful include Gestagens, oestroprogestogens, progestogens, clomiphene citrata, and a GnRH analog with a depot action known as leuprorelin (D-Leu6-Pro9-NH-Ethylamide) or Goserelin depot.

B. Excipients and Carriers

Suitable carriers or excipients can enhance the physical and chemical stability of the formulation or enhance its aesthetic properties. Suitable excipients include, but are not limited to, emulsifiers, diluents, surfactants, solubility enhancers, suspending agents, anti-oxidants, chelating agents, emollients, humectants, pH modifying agents, lipid bilayer disrupting agents, preservatives, thickening agents, viscosity modifying agents, vitamins and other skin nutrients, and combinations thereof.

Suitable emulsifiers include, but are not limited to, straight chain or branched fatty acids, polyoxyethylene sorbitan fatty acid esters, sorbitan fatty acid esters, propylene glycol stearate, glyceryl stearate, polyethylene glycol, fatty alcohols, polymeric ethylene oxide-propylene oxide block copolymers, and combinations thereof.

Diluents may be included in the formulations to dissolve, disperse or otherwise incorporate the carrier. Examples of diluents include, but are not limited to, water, buffered aqueous solutions, organic hydrophilic diluents, such as monovalent alcohols, and low molecular weight glycols and polyols (e.g. propylene glycol, polypropylene glycol, glycerol, butylene glycol).

Suitable surfactants include, but are not limited to, anionic surfactants, non-ionic surfactants, cationic surfactants, and amphoteric surfactants. Examples of anionic surfactants include, but are not limited to, ammonium lauryl sulfate, sodium lauryl sulfate, ammonium laureth sulfate, sodium laureth sulfate, alkyl glyceryl ether sulfonate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium and ammonium salts of coconut alkyl triethylene glycol ether sulfate; tallow alkyl triethylene glycol ether sulfate, tallow alkyl hexaoxyethylene sulfate, disodium N-octadecylsulfosuccinnate, disodium lauryl sulfosuccinate, diammonium lauryl sulfosuccinate, tetrasodium N-(1,2-dicarboxyethyl)-N-octadecylsulfosuccinnate, diamyl ester of sodium sulfosuccinic acid, dihexyl ester of sodium sulfosuccinic acid, dioctyl esters of sodium sulfosuccinic acid, docusate sodium, and combinations thereof.

Examples of nonionic surfactants include, but are not limited to, polyoxyethylene fatty acid esters, sorbitan esters, cetyl octanoate, cocamide DEA, cocamide MEA, cocamido propyl dimethyl amine oxide, coconut fatty acid diethanol amide, coconut fatty acid monoethanol amide, diglyceryl diisostearate, diglyceryl monoisostearate, diglyceryl monolaurate, diglyceryl monooleate, ethylene glycol distearate, ethylene glycol monostearate, ethoxylated castor oil, glyceryl monoisostearate, glyceryl monolaurate, glyceryl monomyristate, glyceryl monooleate, glyceryl monostearate, glyceryl tricaprylate/caprate, glyceryl triisostearate, glyceryl trioleate, glycol distearate, glycol monostearate, isooctyl stearate, lauramide DEA, lauric acid diethanol amide, lauric acid monoethanol amide, lauric/myristic acid diethanol amide, lauryl dimethyl amine oxide, lauryl/myristyl amide DEA, lauryl/myristyl dimethyl amine oxide, methyl gluceth, methyl glucose sesquistearate, oleamide DEA, PEG-distearate, polyoxyethylene butyl ether, polyoxyethylene cetyl ether, polyoxyethylene lauryl amine, polyoxyethylene lauryl ester, polyoxyethylene lauryl ether, polyoxyethylene nonylphenyl ether, polyoxyethylene octyl ether, polyoxyethylene octylphenyl ether, polyoxyethylene oleyl amine, polyoxyethyelen oleyl cetyl ether, polyoxyethylene oleyl ester, polyoxyethylene oleyl ether, polyoxyethylene stearyl amine, polyoxyethylene stearyl ester, polyoxyethylene stearyl ether, polyoxyethylene tallow amine, polyoxyethylene tridecyl ether, propylene glycol monostearate, sorbitan monolaurate, sorbitan monooleate, sorbitan monopalmitate, sorbitan monostearate, sorbitan sesquioleate, sorbitan trioleate, stearamide DEA, stearic acid diethanol amide, stearic acid monoethanol amide, laureth-4, and combinations thereof.

Examples of amphoteric surfactants include, but are not limited to, sodium N-dodecyl-ÿ-alanine, sodium N-lauryl-ÿ-iminodipropionate, myristoamphoacetate, lauryl betaine, lauryl sulfobetaine, sodium 3-dodecyl-aminopropionate, sodium 3-dodecylaminopropane sultanate, sodium lauroamphoacetate, cocodimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, oleamidopropyl betaine, coca dimethyl sulfopropyl betaine, stearyl dimethyl sulfopropyl betaine, lauryl dimethyl sulfoethyl betaine, lauryl bis-(2-hydroxyethyl) sulfopropyl betaine, and combinations thereof.

Examples of cationic surfactants include, but are not limited to, behenyl trimethyl ammonium chloride, bis(acyloxyethyl) hydroxyethyl methyl ammonium methosulfate, cetrimonium bromide, cetrimonium chloride, cetyl trimethyl ammonium chloride, cocamido propylamine oxide, distearyl dimethyl ammonium chloride, ditallowdimonium chloride, guar hydroxypropyltrimonium chloride, lauralkonium chloride, lauryl dimethylamine oxide, lauryl dimethylbenzyl ammonium chloride, lauryl polyoxyethylene dimethylamine oxide, lauryl trimethyl ammonium chloride, lautrimonium chloride, methyl-1-oleyl amide ethyl-2-oleyl imidazolinium methyl sulfate, picolin benzyl ammonium chloride, polyquaternium, stearalkonium chloride, sterayl dimethylbenzyl ammonium chloride, stearyl trimethyl ammonium chloride, trimethylglycine, and combinations thereof.

Suitable solubility enhancing agents include solvents such as water; diols, such as propylene glycol and glycerol; mono-alcohols, such as ethanol, propanol, and higher alcohols; DMSO; dimethylformamide; N,N-dimethylacetamide; 2-pyrrolidone; N-(2-hydroxyethyl) pyrrolidone, N-methylpyrrolidone, 1-dodecylazacycloheptan-2-one and other n-substituted-alkyl-azacycloalkyl-2-ones and other n-substituted-alkyl-azacycloalkyl-2-ones (azones).

Suitable suspending agents include, but are not limited to, alginic acid, bentonite, carbomer, carboxymethylcellulose and salts thereof, hydroxyethylcellulose, hydroxypropylcellulose, microcrystalline cellulose, colloidal silicon dioxide, dextrin, gelatin, guar gum, xanthan gum, kaolin, magnesium aluminum silicate, maltitol, triglycerides, methylcellulose, polyoxyethylene fatty acid esters, polyvinylpyrrolidone, propylene glycol alginate, sodium alginate, sorbitan fatty acid esters, tragacanth, and combinations thereof.

Suitable antioxidants include, but are not limited to, butylated hydroxytoluene, alpha tocopherol, ascorbic acid, fumaric acid, malic acid, butylated hydroxyanisole, propyl gallate, sodium ascorbate, sodium metabisulfite, ascorbyl palmitate, ascorbyl acetate, ascorbyl phosphate, Vitamin A, folic acid, flavons or flavonoids, histidine, glycine, tyrosine, tryptophan, carotenoids, carotenes, alpha-Carotene, beta-Carotene, uric acid, pharmaceutically acceptable salts thereof, derivatives thereof, and combinations thereof.

Suitable chelating agents include, but are not limited to, EDTA, disodium edetate, trans-1,2-diaminocyclohexane-N,N,N′,N′-tetraaceticacid monohydrate, N,N-bis(2-hydroxyethyl)glycine, 1,3-diamino-2-hydroxypropane-N,N,N′,N′-te-traacetic acid, 1,3-diaminopropane-N,N,N′,N′-tetraacetic acid, ethylenediamine-N,N′-diacetic acid, ethylenediamine-N,N′-dipropionic acid, ethylenediamine-N,N′-bis(methylenephosphonic acid), N-(2-hydroxyethypethylenediamine-N,N′,N′-triacetic acid, ethylenediamine-N,N,N′,N′-tetrakis(methylenephosponic acid), O,O′-bis(2-aminoethyl)ethyleneglycol-N,N,N′,N′-tetraacetic acid, N,N-bis(2-hydroxybenzyl)ethylenediamine-N,N-diacetic acid, 1,6-hexamethylenediamine-N,N,N′,N′-tetraacetic acid, N-(2-hydroxyethyl)iminodiacetic acid, iminodiaeetic acid, 1,2-diaminopropane-N,N,N′,N′-tetraacetic acid, nitrilotriacetic acid, nitrilotripropionic acid, nitrilotris(methylenephosphoric acid), 7,19,30-trioxa-1,4,10,13,16,22,27,33-octaazabicyclo[11,11,11] pentatriacontane hexahydrobromide, triethylenetetramine-N,N,N′,N″,N′″,N′″-hexaacetic acid, and combinations thereof.

Suitable emollients include, but are not limited to, myristyl lactate, isopropyl palmitate, light liquid paraffin, cetearyl alcohol, lanolin, lanolin derivatives, mineral oil, petrolatum, cetyl esters wax, cholesterol, glycerol, glycerol monostearate, isopropyl myristate, lecithin, and combinations thereof thereof. Additional emollients are well known, and listings can be found can be found in reference books, for example under “Skin Conditioning Agents-Emollient” and “Skin Conditioning Agents-Occlusive” in the “CFTA Cosmetic Ingredient Handbook”, copyright 1988 by the Cosmetics, Toiletries and Fragrance Association of Washington, D.C.

Suitable humectants include, but are not limited to, glycerin, butylene glycol, propylene glycol, sorbitol, triacetin, and combinations thereof.

The compositions described herein may further contain a pH modifying agent including, but are not limited to, sodium hydroxide, citric acid, hydrochloric acid, acetic acid, phosphoric acid, succinic acid, sodium hydroxide, potassium hydroxide, ammonium hydroxide, magnesium oxide, calcium carbonate, magnesium carbonate, magnesium aluminum silicates, malic acid, potassium citrate, sodium citrate, sodium phosphate, lactic acid, gluconic acid, tartaric acid, 1,2,3,4-butane tetracarboxylic acid, fumaric acid, diethanolamine, monoethanolamine, sodium carbonate, sodium bicarbonate, triethanolamine, and combinations thereof.

Suitable lipid bilayer disrupting agents include fatty acids such as linoleic acid, capric acid, lauric acid, and neodecanoic acid, which can be in a solvent such as ethanol or propylene glycol.

Preservatives can be used to prevent the growth of fungi and other microorganisms. Suitable preservatives include, but are not limited to, benzoic acid, butylparaben, ethyl paraben, methyl paraben, propylparaben, sodium benzoate, sodium propionate, benzalkonium chloride, benzethonium chloride, benzyl alcohol, cetypyridinium chloride, chlorobutanol, phenol, phenylethyl alcohol, thimerosal, and combinations thereof.

To overcome some of the problems with delivery of the drug, e.g., transdermal delivery, that are associated with transport across the dermal layers (“percutaneous absorption”), physiologically active agents are commonly formulated with one or more dermal penetration enhancers (Finnin and Morgan, J. Pharm. Sci., Vol 88, No. 10, October 1999, pp 955-958) which are often lipophilic chemicals that readily partition into the stratum corneum whereupon they exert their effects on improving the transport of drugs across the skin barrier.

Suitable penetration enhancers include urea, (carbonyldiamide), imidurea, N,N-diethylformamide, N-methyl-2-pyrrolidine, 1-dodecal-azacyclopheptane-2-one, calcium thioglycate, 2-pyyrolidine, N,N-diethyl-m-toluamide, alcohols such as jojoba alcohol or lecithin, oleic acid and its ester derivatives, such as methyl, ethyl, propyl, isopropyl, butyl, vinyl and glycerylmonooleate, sorbitan esters, such as sorbitan monolaurate and sorbitan monooleate, other fatty acid esters such as isopropyl laurate, isopropyl myristate, isopropyl palmitate, diisopropyl adipate, propylene glycol monolaurate, propylene glycol monooleatea and non-ionic detergents such as BRIJ® 76 (stearyl poly(10 oxyethylene ether), BRIJ® 78 (stearyl poly(20)oxyethylene ether), BRIJ® 96 (oleyl poly(10)oxyethylene ether), and BRIJ® 721 (stearyl poly (21) oxyethylene ether) (ICI Americas Inc. Corp.).

The concentration of the penetration enhancer is typically from about 1% to about 10% by weight of the formulation.

In certain embodiments, the drug or drugs are present at about 0.0001 to about 10% by weight of the entire formulation, more typically about 0.001 to 1% by weight and in particular about 0.01 to 0.5% by weight.

### II. Methods of Administration

The formulations are administered topically as needed. The formulations are preferably administered locally at or adjacent to the area to be treated, for example, the skin over disturbed subcutaneous connective fatty tissue. As used herein, “affected area” refers to the skin and its surrounding environs. As used herein, “systemically” refers to the circulatory system, and regions outside the spaces described above.

1-9. (canceled)

10. A method for decreasing the amount or appearance of subcutaneous connective fatty tissue comprising applying to the skin at the site of the fatty tissue an effective amount of the formulation comprising a testosterone analog or gonadotrophin releasing hormone analog in a topical carrier for administration of an effective amount to the skin at an area containing disturbed subcutaneous connective fatty tissue.
